(19) United States
(12) Patent Application Publication
(10) Pub. No.: US 2025/0378240 A1
(43) Pub. Date: Dec. 11, 2025

(54) SMART BIO-INSPIRED MATERIAL DESIGN PLATFORM

(71) Applicants: Chih-Yung Chien, Melbourne (AU); NATIONAL CHENG KUNG UNIVERSITY, Tainan (TW)

(72) Inventors: Chi-Hua YU, Tainan (TW); Bor-Yanan Tseng, Tainan (TW); Yu-Wen Chen, Tainan (TW); Chuin-Shan Chen, Tainan (TW); Tzu-Ching Chan, Tainan (TW)

(21) Appl. No.: 19/244,005

(22) Filed: Jun. 20, 2025

Related U.S. Application Data
(63) Continuation-in-part of application No. PCT/AU2022/051540, filed on Dec. 20, 2022.

(51) Int. Cl. G06F 30/27; B29C 64/386; B33Y 50/00; G06F 30/23

(52) U.S. Cl. CPC G06F 30/27; B29C 64/386; G06F 30/23; B33Y 50/00

(57) ABSTRACT

The present invention discloses a smart bio-inspired material design platform to satisfy multi-objective material design featuring complex microstructure for the future. the platform sets mechanical properties of a simulative material element via establishing a reduced model. A distribution of the simulative material element is simulated so as to output a material simulative parameter. A deep learning framework is combined in the platform for computing and evaluating an optimal material design that meets a target material parameter. Specifically, the reduced model can be based on data provided by any test of material mechanical properties, and the deep learning framework evaluates whether a biomimetic material design meets demand of the optimal target material parameter according to a standardized reward function model. The platform is applicable to multi-objective simulative material design, and is greatly potential for futuristic applications.

### CROSS-REFERENCE TO RELATED APPLICATION

This application is a continuation-in-part of PCT Application No. PCT/AU2022/051540, entitled SMART BIO-INSPIRED MATERIAL DESIGN PLATFORM, filed on Dec. 20, 2022, the disclosure of which is hereby incorporated by reference.

### FIELD OF THE INVENTION

The present invention relates to bio-inspired material design, and integrates artificial intelligence technology. In particular, deep learning is applied for simulation of the bio-inspired material structure.

### BACKGROUND OF THE INVENTION

With development of technologies, application of materials and structures in various fields and industries is different. Using a single structure or material for designing and application does not meet demands for growing complexity and precision, which is fundamental for manufacturing of high-added value. In emergence of 3D printing, materials of complex structure can be manufactured in a rapid and precise manner, and such technology has been frequent applied to material research in various industries.

Taking aerospace technology for example, requirements of spacecraft parts and components are increasing. To ensure the safety of a spacecraft, structural stability of parts and components are required at the meantime of increasing traveling speed of the spacecraft. Upon energy saving issues, considering that fuel consumption and carbon emission depends on mass of the spacecraft, and therefore material design will focus on lightweight, solidity and durability.

As for mechanical processing, requirements of high precision and high acceleration for a machine tool become more and more difficult to be satisfied by conventional metal-made machine tool. Miniaturizing of the movement parts in a machine tool requires high precision, low margin tolerance along with high tenacity and rigidity. Material design of the movement parts to meet above requirements will aim to complexity of strain force that the movement parts may confront with.

With human civilization progress, sport industry draws more and more attentions. To meet demand of sport gears from various perspectives, the main rhythm of sport industry will be providing sport gears of durability, lightweight and comfort. For user comfort during exercise, a new type of material to absorb impact force or reaction force generated during exercise will be a decisive factor. Wearable device, such as sneakers, mountain boots, swimming webs, helmets or armors, needs to endure stresses from limbs during exercise, or shocking force when impact with other equipment or field surface. The force of impact has more than a single source, and previous design of single material and single structure is difficult to meet the requirements above.

Currently, materials of higher “specific stiffness” or “specific strength” are used for improving new material design, or material distribution is optimized by analyzing structural loading compacity based on a particular material. In addition to structural enhancement and loading compacity analysis in traditional material science, material design by mimicking biostructure in nature is also a field in rapid growth. Living creatures have developed biostructures of excellent mechanical properties after a long time of evolution in confront with complex and changeable natural environment. The biostructures can be exemplified by the structural stability and strength of bee hives, stiffness and strength of spider web, or outstanding fluid mechanical properties and waterproofness produced by small teeth on shark skin surface. Biomimetic microstructure combining 3D print for material design of superb functional properties including high strength, high energy absorption or lightweight is promising to meet future industrial demands.

Thus, a new-type, data-driven and multi-dimensional finite element simulated material and a platform for complex structure material design is in urgent need. Such a platform will realize material design for complex microstructure required for various properties and fields so as for applications in aerospace, military, automobile, bulletproof coating or sport industries.

### SUMMARY OF THE INVENTION

To solve the above technical issues encountered during biomimetic material structure design, it requires to overcome a wide spectrum of parameters involved in complex structure, such as porosity, porous conformation, strain force, reaction force or strain energy. In order to perform structural design with the material design model using a diversity of parameters and capable of processing the corresponding magnitude of parameters, the present invention takes advantages of metastructure model to generate multiple types of microstructures for analysis, and materials of different porous microstructures are printed by 3D printing for experiment to obtain fundamental material parameters. These parameters, in combination of finite element simulation, are then contributed to artificial intelligence training for material design.

The present invention discloses a smart bio-inspired material design platform comprising a material distribution simulating module configured to execute a target material distribution simulation so as to obtain a material simulative parameter; a reinforcement learning module, communicating to the material distribution simulating module, configured with a deep learning framework for executing a reward function model to compute a reward value of an iterative distribution simulant of another target material according to an optimal target parameter (P), and evaluating whether the iterative distribution simulant meet the optimal target parameter (P), wherein the target material comprises a stiff material and a soft material, and each material is set with different material model coefficient combinations, and the optimal target parameter (P) is set by referring to the material simulative parameter comprising strain energy (SE), reaction force (RF), average mises stress (ST) or a combination of two or more thereof.

In some embodiments, the reward function model comprises modifying an initial distribution simulant into the iterative distribution simulant so as to obtain a variable quantity (D) based on the deep learning framework; assigning the initial distribution simulant an initial reward value (q1) and assigning the iterative distribution simulant an end reward value (q2) so as to compute a standardized reward value (Q); and the deep learning framework evaluates whether the iterative distribution simulant meet the optimal target parameter (P) according to the standardized reward value (Q).

Preferably, the standardized reward value (Q) is computed according to the following formula:

$$Q = [ q 1 q 2 ] * d * P ,$$

wherein d is a standardized coefficient calculated through the following formula:

$$d = σ [ ( D - α ) / β ] ,$$

and wherein α is a mean value of the variable quantity (D), and β is an standard deviation of the variable quantity (D), and σ is a Sigmoid function.

In some embodiments, the smart bio-inspired material design platform further comprises a compression experiment module connecting to the reduced model construction module, comprising an experimental structure generator for generating a metastructure based on a metastructure model, and an structure compressor for compressing the metastructure so as to obtain the compression data, wherein the metastructure is equal to or different from the experimental structure.

Preferably, the material distribution simulating module comprises a finite element simulator configured for generating the initial distribution simulant by arranging the target material according to the optimal target parameter (P).

In one or various embodiments, the smart bio-inspired material design platform comprises a reduced model construction module communicating to the material distribution simulating module for constructing the target material, wherein the reduced model construction module comprises a Master curve generator for generating a Master curve according to a compression data, wherein the compression data comprises Young's modulus, plateau stress and relative density; a material coefficient curve-fitting simulator for curve fitting a selected experimental structure to the Master curve so as to obtain a material coefficient combination and output a material model coefficient combination; and a target material generator for assigning the material coefficient combination to a single element so as to obtain the target material, wherein the material model coefficient combination comprises a stiff material model coefficient combination, a soft material model coefficient combination or a combination thereof.

Preferably, the experimental structure comprises Gyroid, Primitive, F-RD, Fischer-Koch S, Diamond or I-WP, or the experimental structure is generated based on a metastructure model, wherein the metastructure model comprise Triply Periodic Minimal Surface model (TPMS model).

In some preferred embodiments, the smart bio-inspired material design platform further comprises a compression experiment module connecting to the reduced model construction module, comprising an experimental structure generator for generating a metastructure based on a metastructure model, and an structure compressor for compressing the metastructure so as to obtain the compression data, wherein the metastructure is equal to or different from the experimental structure.

Preferably, the experimental structure comprises Gyroid, Primitive, F-RD, Fischer-Koch S, Diamond or I-WP, or the experimental structure is generated based on a metastructure model.

Preferably, the metastructure model comprise Triply Periodic Minimal Surface model (TPMS model).

In other preferred embodiments, the material distribution simulating module comprises a finite element simulator configured for generating a material distribution simulant with the target material so as to generate a rigid compress body, and outputting the material simulative parameter by simulating compression of the rigid compress body upon the material distribution simulant.

Preferably, the deep learning framework comprises Deep Q-Networks.

In various embodiments, the smart bio-inspired material design platform further comprises a material design module communicating to the reinforcement learning module for designing a bio-inspired simulated distribution simulant according to a second optimal target parameter customized by a user, wherein the second optimal target parameter is set by referring to the material simulative parameter comprising strain energy (SE), reaction force (RF), average mises stress (ST) or a combination of two or more thereof.

In another aspect, the present invention discloses a method for designing smart bio-inspired material, comprising executing a target material distribution simulation with a material distribution simulating module so as to obtain a material simulative parameter, wherein the target material comprises a stiff material and a soft material, and each material is set with different material model coefficient combinations; and executing a reward function model with a deep learning framework configured to a reinforcement learning module for computing a reward value, and evaluating whether an iterative distribution simulant meet the optimal target parameter (P), wherein the reward value is computed corresponding to an iterative distribution simulant of another target material according to an optimal target parameter (P), wherein the optimal target parameter is set by referring to the material simulative parameter comprising strain energy (SE), reaction force (RF), average mises stress (ST) or a combination of two or more thereof.

In various embodiments, the reward value computation comprises modifying an initial distribution simulant into the iterative distribution simulant via the reward function model so as to obtain a variable quantity (D) based on the deep learning framework; assigning the initial distribution simulant an initial reward value (q1) and assigning the iterative distribution simulant an end reward value (q2) so as to compute a standardized reward value (Q); and evaluating whether the iterative distribution simulant meet the optimal target parameter (P) according to the standardized reward value (Q) via the deep learning framework.

Preferably, the standardized reward value (Q) is computed according to the following formula:

$$Q = [ q 1 q 2 ] * d * P ,$$

wherein d is a standardized coefficient calculated through the following formula:

$$d = σ [ ( D - α ) / β ] ,$$

and wherein α is a mean value of the variable quantity (D), and β is an standard deviation of the variable quantity (D), σ is a Sigmoid function.

In preferred embodiments, the material distribution simulating comprises generating a material distribution simulant with the target material via a finite element simulator, generating a rigid compress body, and exporting the material simulative parameter by simulating compression of the rigid compress body compressing the material distribution simulant.

More preferably, the initial distribution simulant is generated by a finite element simulator arranging the target material according to the optimal target parameter (P).

In other preferred embodiments, the method further comprises modeling a reduced model comprising generating a Master curve via a Master curve generator according to a compression data, wherein the compression data comprises Young's modulus, plateau stress and relative density; curve fitting a selected experimental structure to the Master curve via a material coefficient curve-fitting simulator so as to obtain a material coefficient combination and exporting a material model coefficient combination; and assigning the material coefficient combination to a single element via a target material generator so as to obtain the target material, wherein the material model coefficient combination comprises a stiff material model coefficient combination, a soft material model coefficient combination or a combination thereof.

Preferably, the experimental structure comprises Gyroid, Primitive, F-RD, Fischer-Koch S, Diamond or I-WP, or the experimental structure is generated based on a metastructure model.

More preferably, the compression data obtaining comprises generating the compression data via a compression experiment module, wherein the compression data generating comprises generating a metastructure according to a metastructure model via an experimental structure generator, and compressing the metastructure via a structure compressor so as to obtain the compress data, wherein the metastructure is equal to or different from the experimental structure.

Preferably, the experimental structure comprises Gyroid, Primitive, F-RD, Fischer-Koch S, Diamond or I-WP, or the experimental structure is generated based on a metastructure model.

More preferably, the metastructure model comprise Triply Periodic Minimal Surface model (TPMS model).

In some preferred embodiments, the method further comprises designing a bio-inspired simulated distribution simulant via a material design module according to a second optimal target parameter customized by a user, wherein the second optimal target parameter is set by referring to the material simulative parameter comprising strain energy (SE), reaction force (RF), average mises stress (ST) or a combination of two or more thereof.

The present invention provides a standardized workflow for material design. As long as the reward function model of the reinforcement learning model is well defined, a corresponding structural design can be efficiently simulated, which satisfies demands of material structural design for various situations.

The reduced model disclosed in the present invention improves subsequent computation of target material distribution by setting material simulative parameters for target material. The reduced model reduces the number of complex structural parameters involved by target material, and diminishes time required for the reinforcement learning model computation.

The smart bio-inspired material design platform disclosed in the present invention utilizes deep learning framework based on artificial intelligence for biomimetic material design. The biomimetic material as designed referring to the simulation result successfully lowers concentration of reaction force and strain force when the biomimetic material is under compression. Such a platform is beneficial for material design involving complex microstructure, and widely applicable for designing sport products including shoe midsole, integrally formed sandals, handle of badminton racket, helmet, golf ball or golf clubs.

### BRIEF DESCRIPTION OF THE DRAWINGS

FIG. 1A is a block chart to illustrate the structure of the bio-inspired material design platform in the present invention. FIG. 1B is a simulative shoe midsole to provide a conceptual illustration of the material distribution simulant. FIGS. 2A to 2B are flowcharts to illustrate the method for designing smart bio-inspired material in various embodiments in the present invention. FIG. 3 demonstrates a particular embodiment of the method for designing smart bio-inspired material in the present invention. FIG. 4 demonstrates the structure of test body in the compression experiment and stress-strain curve generated thereby. FIGS. 5A to 5B illustrate Master curves. FIG. 6 is a flowchart to illustrate the process of material model coefficient curve fitting in a particular embodiment. FIG. 7 is a flowchart to illustrate the process of material simulative parameter curve fitting in a particular embodiment. FIG. 8 is a flowchart to illustrate the executive process of a reward function model and bio-inspired material design by the deep learning framework. FIGS. 9A to 9C are conceptual diagrams of material distribution simulants' stiff-soft materials distribution and simulative diagrams of strain force distribution thereof. FIG. 10 is a conceptual diagram to illustrate the technology integrated in the present invention and corresponding futuristic application thereof.

### DETAILED DESCRIPTION OF THE INVENTION

Hereinafter several examples are used to illustrate the technical connotation of the present invention and the technical effects that are specifically achieved. The purpose is not to limit the scope of protection which should refer to the content contained in the scope of the claims. Improvements or expansions that derives from the technical spirit of the present invention are all within the scope of protection based on claims in the instant application.

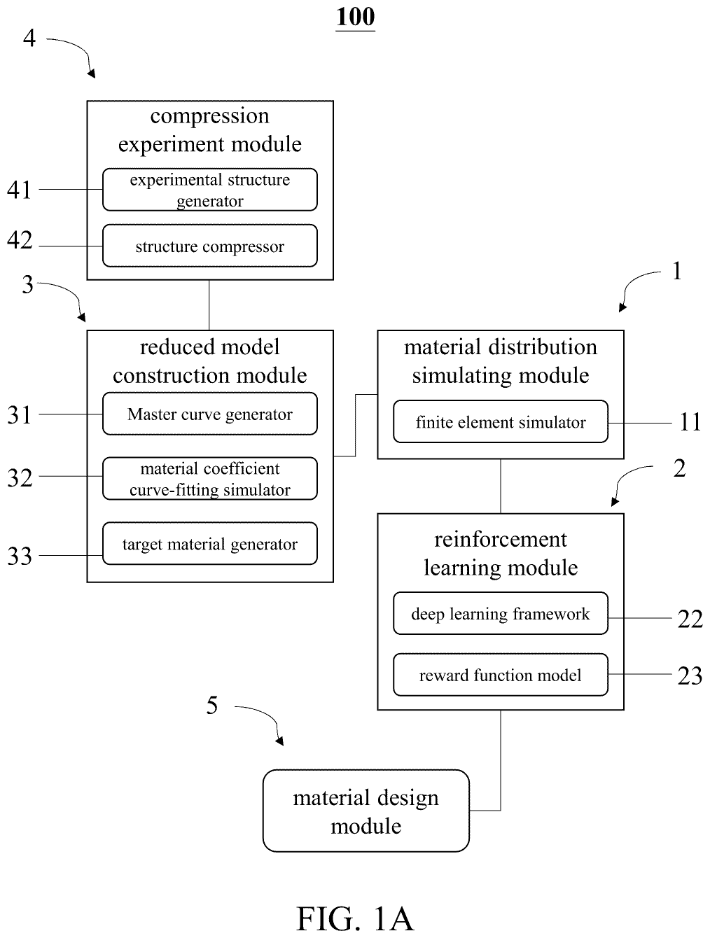

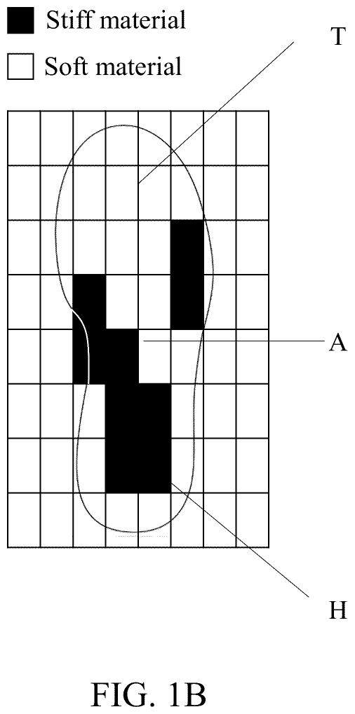

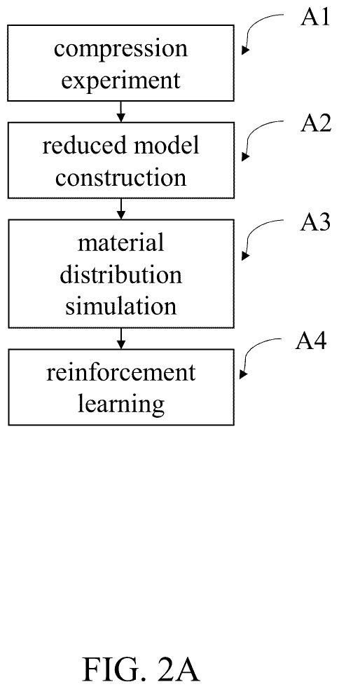

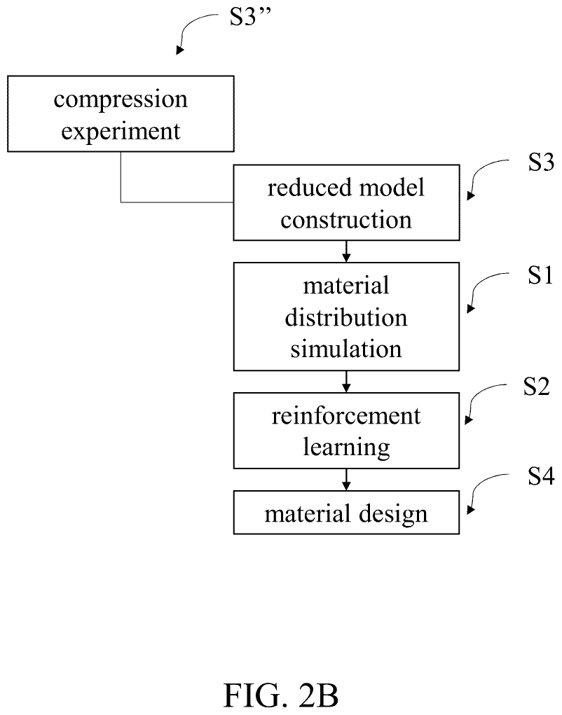

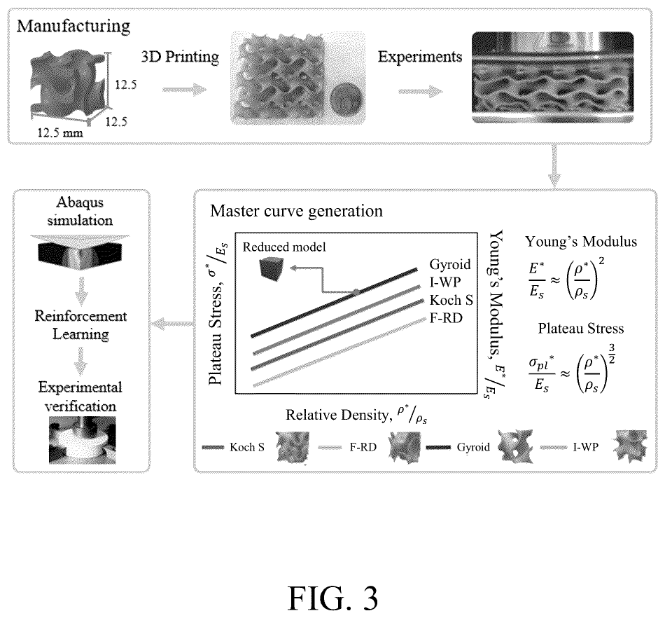

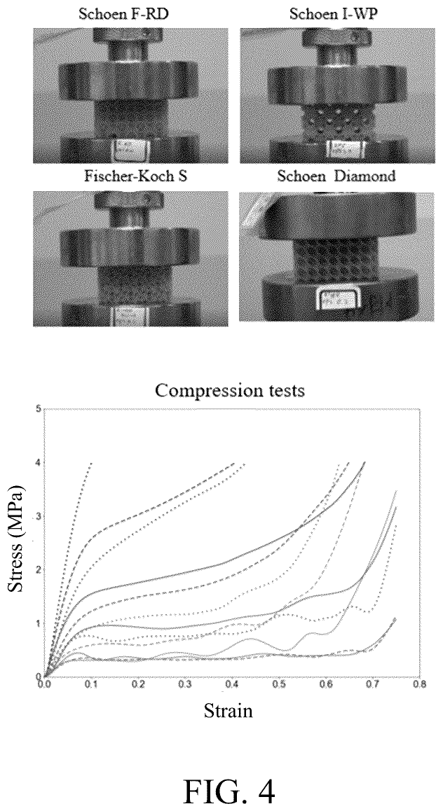

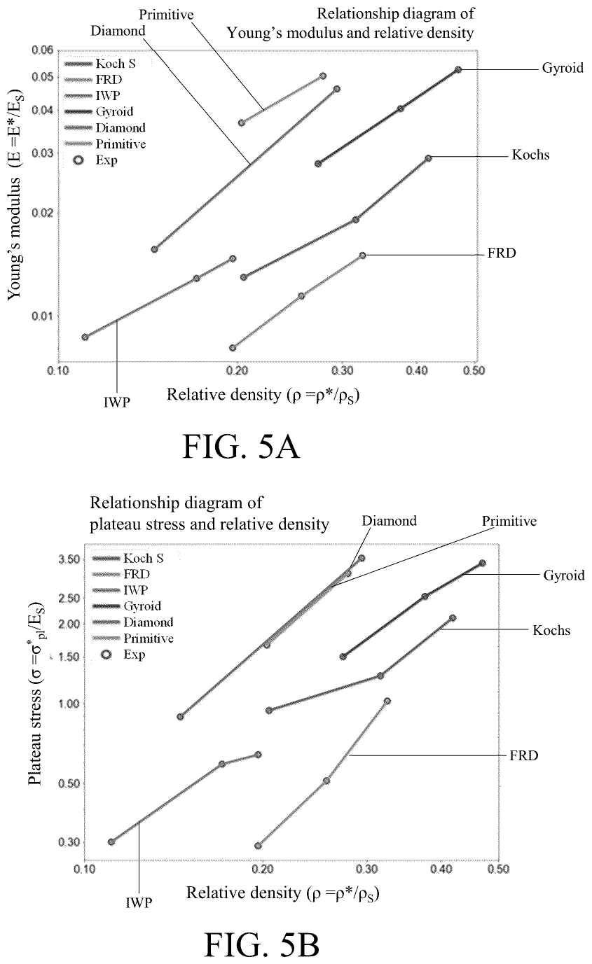

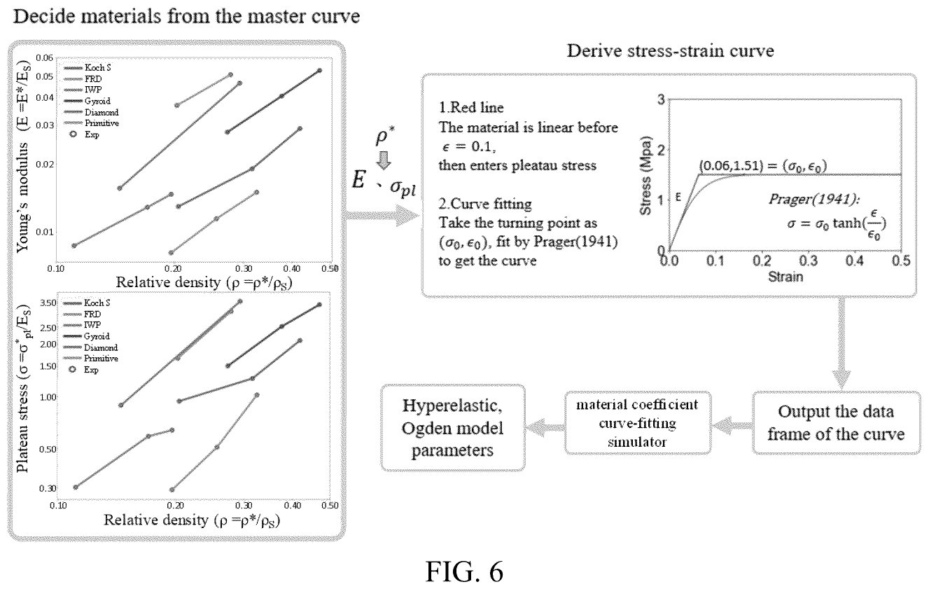

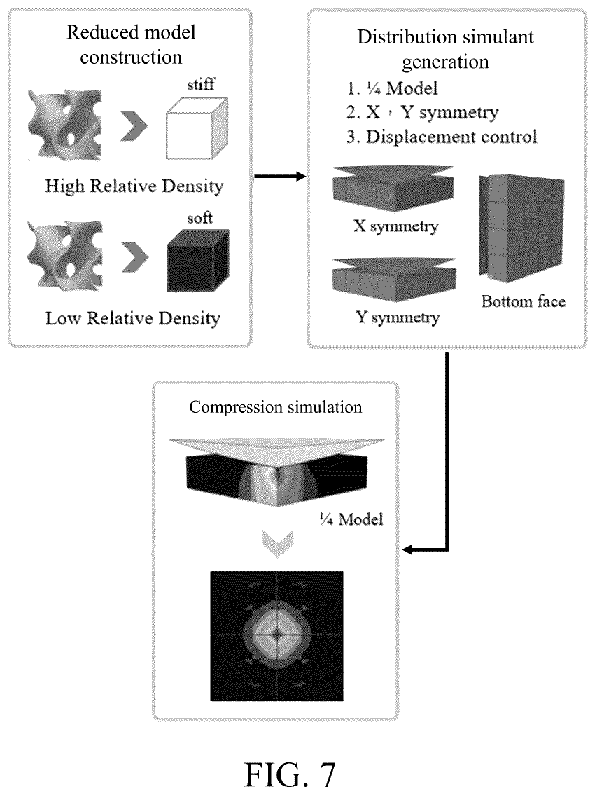

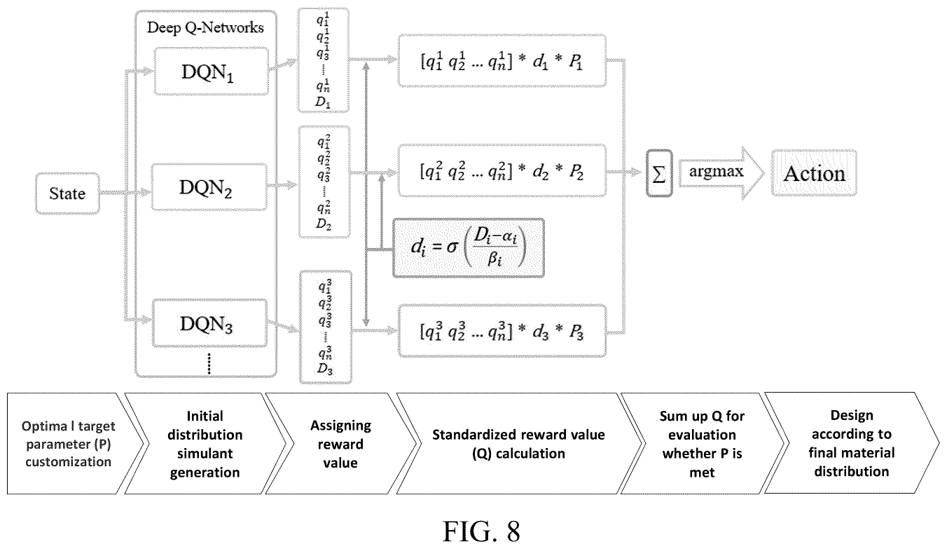

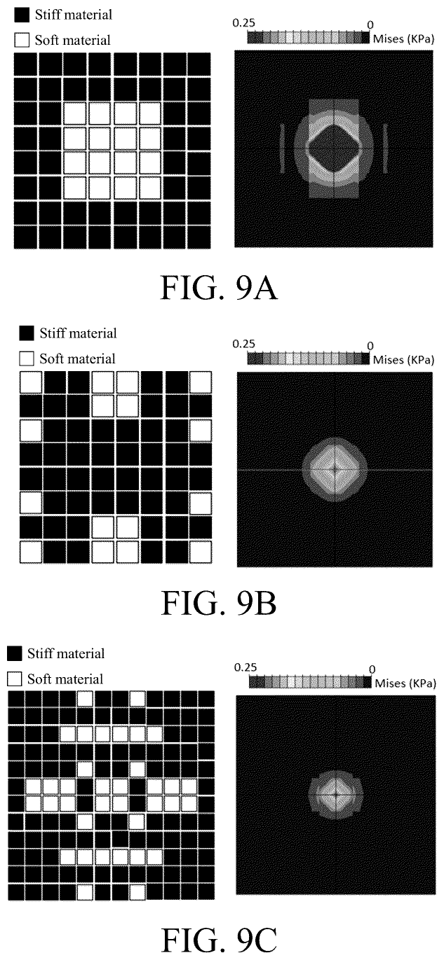

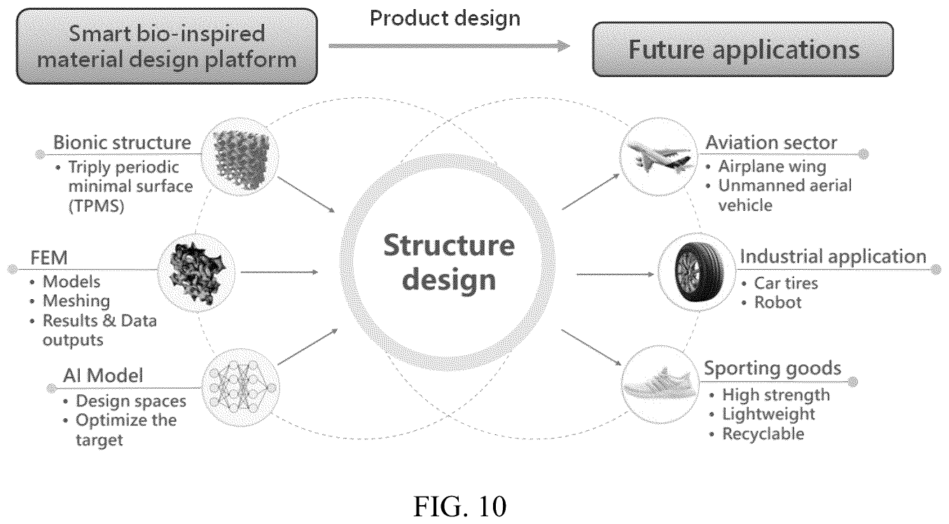

In one aspect, as shown in FIG. 1A, the present invention provides a smart bio-inspired material design platform (100) comprising a material distribution simulating module (1) configured to execute a target material distribution simulation so as to obtain a material simulative parameter, and a reinforcement learning module (2), communicating to the material distribution simulating module, configured with a deep learning framework (21) for executing a reward function model (22) to compute a reward value of an iterative distribution simulant of another target material according to an optimal target parameter (P), and evaluating whether the iterative distribution simulant meets the optimal target parameter (P), wherein the target material comprises a stiff material and a soft material, and each material is set with different material model coefficient combinations; the optimal target parameter (P) is set according to the material simulative parameter comprising strain energy (SE), reaction force (RF), average mises stress (ST) or a combination of two or more thereof.

In one or various embodiments, as shown in FIG. 1A, the material distribution simulating module (1) comprises a finite element simulator (11) configured for generating a material distribution simulant with the target material, generating a rigid compress body, and exporting the material simulative parameter by simulating the rigid compress body compressing the material distribution simulant.

The smart bio-inspired material design platform (100) further comprises a reduced model construction module (3) communicating to the material distribution simulating module (1) for constructing the target material, wherein the reduced model construction module (3) comprises a Master curve generator (31) for generating a Master curve according to a compression data, wherein the compression data comprises Young's modulus, plateau stress and relative density; a material coefficient curve-fitting simulator (32) for curve fitting a selected experimental structure to the Master curve so as to obtain a material coefficient combination and outputting a material model coefficient combination; and a target material generator (33) for assigning the material coefficient combination to a single element so as to obtain the target material, wherein the material model coefficient combination comprises a stiff material model coefficient combination, a soft material model coefficient combination or a combination thereof.

It should be noted that the compression data can be the material mechanical parameter of the target material corresponding to any of the experimental structures. The compression data can be exemplified by Young's modulus, rigidity modulus, elasticity modulus, shear elasticity modulus, strain force, modulus of deformation, strain energy, modulus of strain variable. Specifically, stress refers to applied force loading per unit area, wherein the applied force can be pulling force, pushing force, shear force, bending force or torsion force, and not limited to this. Strain refers to deformation caused by the applied force on an object, such as plastic deformation and elastic limit. The aforementioned material mechanic parameter is not limited to an individual parameter or a group of different parameters. The material mechanic parameter can also be a ratio of two or more parameters as listed hereinabove. For example, a proportional limit is defined by the correlation of stress and strain. Preferably, the compression data is obtained by a compression experiment module (4) which practically performs experimental structure compression via a compression testing machine. The experimental structure can be obtained by 3D printing referring to a simulative solid structure. It can be understood that acquiring the compression data is not limited to the aforementioned method. The material mechanical parameter corresponding to a metastructure having any one of microstructure, biomimetic microstructure and lattice structure can be used to establish the reduced model.

In some preferred embodiments, as shown in FIG. 1A, the compression experiment module (4) connects to the reduced model construction module (31). and the compression experiment module (4) comprises an experimental structure generator (41) for generating a metastructure based on a metastructure model, and an structure compressor (42) for compressing the metastructure so as to obtain the compression data, wherein the metastructure is equal to or different from the experimental structure. The metastructure refers to a cellular structure, particularly a lattice structure which demonstrates mechanic properties including high specific stiffness, specific strength or shock energy absorption. Through microstructural design of the lattice structure, adjustment of the mechanical properties can be realized so as to satisfy material designs of various need. For example, adjusting coefficient of thermal expansion (CTE) and Poisson's ratio (PR) to be positive, null or negative can be a strategy of conformational changes. Specifically speaking, the metastructure is not limited thereto. Any solid structure having a regular microstructure and obtained according to a metastructure model via its mathematic algorithm can be used by the platform (100) for bio-inspired material design in the present invention, and the basic metastructure unit can be exemplified by Idealized foam, Kelvin, Cubic or Octet.

Particularly, the metastructure model comprises a Triply Periodic Minimal Surface model (TPMS model). The TPMS model can be used for generating a composite cellular structure having regularity.

It should be noted that the simulative compression in the present invention is based on a finite element model, and wherein the finite element model can be ABAQUS, ANSYS, OpenFOAM, SimScale, Autodesk CFD or RoboLogix, but not limited thereto. The simulative compression comprises setting a compression center of the material distribution simulant, and the compression center can be the geometric center (also known as centroid), or any of the surface points of the material distribution simulant. For instance, as shown in FIG. 1B, the material distribution simulant is exemplified by a shoe midsole simulant whose compression center can be set according to the specific demand of a shoe midsole. In one example, the demand is high supporting capacity of an arch (A), and the compression center is set at the A site of an arch. In another example, the demand is to reduce the vibration of a foot plantar with a high energy absorption design, so the compression center is set at the H site of a heel. The compression center setting is not limited to the examples as mentioned above, and the compression center can be customized according to users' need. For example, the simulative compression center can be set in reference to the gravitational center or the mass center of a user during walking, running or standing. On the other hand, the simulative compression center can be set by referring to the force-exerting state when the exerted force of heel is greater than that of the arch, during the aforementioned activities. The simulative compression center can be set according to various situations so as to satisfy multi-target demands. Similarly, the compression center setting can be a single point or a plurality of points so as to meet demand(s) of complex pull-strain structure of other materials such as a racket head. Material design of a racket head is required to considering pull or reaction forces during racket stringing or striking a shuttlecock. Then, compression experiment simulation is performed with dynamic explicit via the finite element model. Two selected material model coefficient combinations are assigned to single elements to be a soft material and a stiff material, and a plurality of soft materials and a plurality of stiff materials are further customized to be arranged into a material distribution simulant. Ratio of the soft materials to the stiff materials variates as demand changes. The material distribution simulant can be a random polygon or a random asymmetric shape, such as a triangle, a regular tetragon, a pentagon, or a hexagon, but not limited to this. The asymmetric shape can be a planar shape such as a foot plantar or a palm, or the asymmetric shape can be a solid object such as a racket, a bat, a golf clubs, an engine bearing, a piston, a tire, a tire frame, a hydraulic valve body, a plane outer shell, or a plane wing, and not limited thereto. Subsequently, after the material distribution simulant customization is completed, perimetric conditions and a given displacement control are also customized. The material distribution simulant is immobilized, and the rigid compress body moves downwards and compresses the material distribution simulant to a specific compression time so as to export the material simulative parameter including strain energy (SE), reaction force (RF) or average mises stress (ST), wherein the speed of rigid compress body moving downward for compression can be a uniform speed, a speed of uniform acceleration, or a speed of inconstant acceleration. It should be noted that fixing the proportion of soft materials and stiff materials aims to optimize material arrangement with the least material consumption. For saving time of simulation computing, in some preferred embodiments, the compression experimental simulation is executed with customization of symmetric simulation.

Disclosed hereinafter is a particular embodiment of the smart bio-inspired material design platform (100). Shown in FIG. 2A is a flowchart to illustrate the basic working concept of the platform (100). Firstly, as illustrated in step A1, a compression experiment is performed to establish a 3D model of the experimental structure with the experimental structure generator (41) according to a program, and the experimental structure is manufactured by 3D printing for compression experiment with the structure compressor (42). Secondly, as shown is step A2, a reduced model is constructed according to the compression data obtained from the compression experiment, wherein the compression data is postprocessed by the Master curve generator (31) so as to generate a Master curve corresponding to any of the compression data. The Master curve as described herein is the correlation of the experimental structure's relative density and intensity. Subsequently, the material coefficient curve-fitting simulator (32) reads the Master curve for curve fitting the experimental structure so as to obtain two material coefficient combinations, and the target material generator (33) assigns the material coefficient combinations to single voxels as a soft material and a stiff material, which renders the voxel have complex structural features. Thirdly, as shown in step A3, simulating material distribution is performed with the finite element simulator (11). The finite element simulator (11) is used for simulating the process of compressing target material so as to obtain a strain distribution. Fourthly, as shown in step A4, reinforcement learning is carried out with the deep learning framework (21) performing the reinforcement model. The material simulative parameter obtained from material distribution simulation is used by the deep learning framework (21) for matching the material distribution which meets requirements of the target design, wherein the deep learning framework (21) performs the reward function model (22) for calculating a reward value of the corresponsive target material distribution. The material design matching the target material distribution is then 3D printed for experimental tests.

In one exemplary embodiment, as shown in FIG. 3, a unit microstructure corresponding to each form is generated according to the mathematical formula of triple periodic minimum surface (TPMS), and experimental test bodies with dimensions of 50×50×25 mm in length, width and height is 3D printed. Subsequently, a compression experiment is performed with a universal testing machine.

In the exemplary embodiment, Gyroid, Primitive, F-RD, Fischer-Koch S, Diamond, or I-WP is selected as the unit microstructure of experimental test bodies. Three different relative densities corresponding to each unit microstructure are selected for compression experiment. Specifically, the experimental test bodies is lain flat on the compression platform of the universal testing machine. Height of the experimental test bodies is compressed to 75% of the initial height. Practically, an experimental test bodies of 25 mm in height is compressed to be 18.75 mm in height. The compression rate is customized to 1.5 mm per minute, and the experimental process is recorded in video. Through compression experiment, a stress-strain curve corresponding to each experimental test bodies can be obtained. As shown in FIG. 4 are stress-strain curves of the experimental test bodies in the exemplary embodiment. The compression data of each experimental test bodies is acquired through the stress-strain curves. The compression data includes Young's modulus, Plateau stress and relative density, wherein the relative density is calculated via the following formula (1):

$$relative density = \frac{subject mass}{subject solid volume \times Material density} \qquad ( 1 )$$

After the compression data of these experimental test bodies is obtained, the behavior curve of the porous material when compressed are further deduced with Ashby et al. so that a Master curve of each experiment test body can be obtained. The properties of structural materials having various relative densities can be obtained from these Master curves. As the relative density changes, the experimental test bodies would have structures with various wall thicknesses. There are so many types of structures that it is difficult to print them all out for experiments. Experiment time is largely saved in the present invention through determining the mechanical properties of structural materials from the Master curves.

In the exemplary embodiment, as shown in FIG. 5A, the relationship diagram corresponding to the relative density and Young's modulus of each experimental test body can be obtained through formula (2). Shown in FIG. 5B is a relationship diagram established through formula (3), and each curve corresponds to the relative density and plateau stress of each experimental test body.

$$\frac{{E}^{*}}{{E}_{s}} \approx {( \frac{{ρ}^{*}}{{ρ}_{s}} )}^{2} \qquad ( 2 )$$

$$\frac{{σ}_{pl}^{*}}{{E}_{s}} \approx {( \frac{{ρ}^{*}}{{ρ}_{s}} )}^{\frac{3}{2}} \qquad ( 3 )$$

The individual meanings of the symbols involved in the formula (2) and (3) are as follows, E* is the Young's modulus of the experimental test body, E_{S} is the Young's modulus of the experimental test body material, ρ* is the relative density of the experimental test body, ρ_{S} is the relative density of the experimental test body material, and 94 ₚ₁* is the stress of the experimental test body. The parameters of each experiment are substituted into the formula and the logarithm is fetched. The curve trend and position of each experimental test body on the Master curve can be observed, and wherein the E_{S} is the Young's modulus of the experimental test body divided by the Young's modulus of curing resin so as to purely calculate the strength effect of the structure. This is to determine the strength of the corresponding structural material by relative density, regardless of using any type of materials.

As shown in FIG. 6, after selecting the experimental structure for fitting material coefficient, the corresponding material coefficient can be obtained from the Master curve. It should be noted, the experimental structure for material coefficient fitting can be two or more the same or different experimental structures. After specific material coefficients such as Young's modulus, plateau stress and relative density are obtained, they are substituted into the formula disclosed by Prager. The stress-strain curve of the experimental test body is simplified to an ideal stress-strain curve, from which several pieces of data are randomly selected and put into material coefficient curve-fitting simulator (32) to perform parameter fitting. In this example, ABAQUS is used for parameter fitting, and a material model coefficients combination of Hyperelastic or Ogden model is obtained, and the material model coefficients combination represents the deformation behavior and strength of the selected experimental structure in the Master curve.

Subsequently, the material model coefficients combination is assigned to a single element through the target material generator (33) so that the single element has the mechanical properties of the corresponding experimental structure, which is referred to as a reduced model (RM) here. After obtaining these reduced models which are the target materials required for subsequent bio-inspired material design, two parts can be constructed with the ABAQUS model in the finite element simulator (11). The first part is the material distribution simulant composed of the target materials. In this example, the material distribution simulant is a one fourth square flat plate of totally 16 single elements divided into 4×4 in length and width. The second part is a rigid body compression object, in this example, the rigid body compression object is a rigid body hemispherical plate.

Further, the center point of the square plate is set as a reference point, and ABAQUS is used to perform compression test simulation. Two material model coefficient combinations are selected and assigned to the single elements as a soft material and a stiff material, respectively. Within the 16 soft and stiff materials in the plate, the ratio of soft materials to stiff materials is 3:1. Then, the boundary conditions is set with a given displacement control, the square plate is set to be x-axis symmetric and y-axis symmetric, and the square plate bottom is fixed. The rigid body hemispherical plate compresses downwards the square plate at a speed of 0.5 mm/s, and the total compression time is 1 second. It should be noted, setting the one fourth square plate for symmetric simulation is to optimize the material distribution with a limited material consumption. Because the compression point of the rigid body hemispherical plate is located at the center of the square plate, and the material design space is reduced to one fourth by symmetric simulation, the calculation time can be greatly reduced. The process for material simulant compression by the finite element simulator (11) is illustrated in FIG. 7. In the grid setting, the grid form of the square plate is Hex, but it is not limited thereto. The grid form can also be any polygon, such as triangle, rectangular, pentagonal, or any metastructure lattice unit. In addition, the grid form of the rigid body hemispherical plate is Quad-dominated, and it is not limited thereto. Moreover, in this example, in the compression center of the square plate has an Element set for analyzing the average stress of the compressed center.

After the compression simulation of the material distribution simulant through the finite element simulator (11), the material model parameter combination can be exported as the evaluation reference for the follow-up reinforcement learning module (2) to perform deep learning. The user can choose different material model parameter combinations to establish the optimal target parameter (P) according to the material design requirements. For example, the material model parameter combination includes material parameters such as strain energy (SE), reaction force (RF), Average mises stress (ST) and relative density (RD). In this example, the strain energy SE represents the amount of energy absorbed by the material distribution simulant, the stronger the energy absorption capacity, the greater the value of the strain energy SE. The reaction force RF represents the negative acceleration value, reflected by post-processing, of the material distribution simulant in the impact test. The smaller the reaction force RF value is, the smaller the negative acceleration value is. The Average mises stress ST represents the degree of stress concentration of the material distribution simulant during the compression simulation. The relative density RD is the density parameter obtained by dividing the target material that constitutes the material distribution simulant by the curing resin. The lower the relative density RD value, the lower the corresponding material cost.

It can be understood that, the simulative compression adopted in this embodiment is performed with displacement control of the spherical rigid flat plate. Under the same displacement condition, higher force produces higher strain energy. Similarly, the force generated by 5% compression on the stiff material is greater than that of the soft material, so the strain energy caused by the stiff material is also greater. Moreover, since the simulative compression is performed with a spherical rigid flat plate, the larger the displacement by compression in proximity of the compression center, the higher the material simulation parameters such as strain energy and reaction force are exported.

In various embodiments, the smart bio-inspired material design platform (100) calculates the reward value (Q) corresponding to the optimal target parameter (P) through the reward function model (22) so as to evaluate whether the simulated material distribution result satisfies the optimal target parameter (P) customized by the user. Specifically, the reward function model (22) comprises using the deep learning framework (21) to change an initial distribution simulant to the iterative distribution simulant to obtain a variable quantity (D); giving the initial distribution simulant an initial reward value (q1), and the iterative distribution simulant a terminal reward value (q2), and calculating a standardized reward value (Q); the deep learning framework (21) evaluates whether the iterative distribution simulant satisfies the optimal target parameter (P) according to the standardized reward value (Q).

In some preferred embodiments, the standardized reward value (Q) is calculated through the following formula (4):

$$Q = [ q 1 q 2 ] * d * P \qquad ( 4 )$$

wherein the d is a standardized coefficient calculated through the following formula (5):

$$d = σ [ ( D - α ) / β ] \qquad ( 5 )$$

wherein α is a mean value of the variable quantity (D), β is an standard deviation of the variable quantity (D), and σ is a Sigmoid function. It should be noted that d as a standardized coefficient is for standardizing values of multiple design targets so as to establish a comparison range of each target. Such standardization avoids reward value calculation at back end from dissatisfying the optimal target parameter, which results from overemphasis on particular target design.

In the aforementioned embodiment, the initial distribution simulant is generated by the finite element simulator (11) comprising arranging the target material so as to generate the initial distribution simulant according to the optimal target parameter (P).

In the aforementioned embodiment, the deep learning framework mimics the working mechanism of a human neural network. The deep learning framework comprises Multilayer Perceptron, Deep Neural Network (DNN), Convolutional Neural Network (CNN), or Recurrent Neural Network (RNN), and not limited thereto. Preferably, the deep learning framework is a Deep Q-Networks.

The working principle of the reinforcement learning module (2) is exemplarily explained below. Taking Deep Q-Networks as an example, as shown in FIG. 8, bio-inspired material design is performed in the reinforcement learning model based on the material simulative parameters obtained in the aforementioned examples. After the user sets the optimal target parameter (P), ABAQUS generates the initial distribution simulant by arranging the target materials with reduced models. In this example, there are multiple initial distribution simulants marked separately DQN₁, DQN₂, DQN₃ . . . , as the initial state of reinforcement learning. In the subsequent reinforcement learning process, ABAQUS continuously changes the position of soft materials and stiff materials to perform iteratively updated material distribution simulation. The iterative distribution simulant comprises a plurality of iterative distribution simulants, and each iteration produces a distribution simulant composed of target materials with different arrangement and distribution forms from other simulants. Then, Deep Q-Networks decides which action to take regarding these distribution simulations, and the calculation process is multiplied by the decision value (D) representing the amount of change produced between the previous distribution simulant and the next distribution simulant. Hereinafter, the reinforcement learning module (2) assigns a reward value (Q) to each distribution simulant.

Since the setting of the optimal target parameter (P) involves a variety of material simulation parameters, each distribution simulation would be carried out according to multiple material simulation parameters. In order to uniformly compare the distribution simulations corresponding to each time of distribution simulation, the reward value (Q) is standardized with the coefficient d to calculate the standardized reward value (Q) so as to determine which distribution simulant has a greater impact on the design. Also, whether the final iterative distribution simulant meets the optimal target parameter (P) setting would be evaluated according to the standardized reward value (Q). In this example, please continue to refer to FIG. 8, α and β are the mean and the standard deviation of the variable quantity (D), respectively, and σ represents the Sigmoid function. During the training process, distribution simulation of the high standardized reward value (Q) would be recorded, and distribution simulation of the highest standardized reward value (Q) would be selected and summed up. Subsequently, the distribution simulation would be iterated so as to obtain the final target material distribution which is the material distribution design that meets the optimal target parameter (P) setting.

Furthermore, the smart bio-inspired material design platform (100) further comprises a material design module (5) communicating to the reinforcement learning module (2) for designing a bio-inspired simulated distribution simulant according to a second optimal target parameter customized by a user, wherein the second optimal target parameter is customized with the material simulative parameter comprising strain energy (SE), reaction force (RF), average mises stress (ST) or a combination of two or more thereof.

In another aspect, the present invention provides a method for designing smart bio-inspired material, as shown in FIG. 2B, and the method comprises steps as illustrated hereinafter: in step S1, a target material distribution simulation is executed with a material distribution simulating module (1) so as to obtain a material simulative parameter, wherein the target material comprises a stiff material and a soft material set with different material model coefficient combinations; and in step S2, a reward function model (22) is executed under a deep learning framework (21) configured to a reinforcement learning module (2) for computing a reward value, and evaluating whether an iterative distribution simulant meets the optimal target parameter (P), wherein the reward value is computed corresponding to an iterative distribution simulant of another target material according to the optimal target parameter (P), wherein the optimal target parameter is set according to the material simulative parameter comprising strain energy (SE), reaction force (RF), average mises stress (ST) or a combination of two or more thereof.

In one or various embodiments, the reward value computation comprises changing an initial distribution simulant into the iterative distribution simulant under the deep learning framework (21) so as to obtain a variable quantity (D); assigning an initial reward value (q1) to the initial distribution simulant and assigning an end reward value (q2) to the iterative distribution simulant so as to compute a standardized reward value (Q); and evaluating whether the iterative distribution simulant meets the optimal target parameter (P) according to the standardized reward value (Q) under the deep learning framework.

In some preferred embodiments, the standardized reward value (Q) is calculated according to the following formula (4):

$$Q = [ q 1 q 2 ] * d * P \qquad ( 4 )$$

wherein d is a standardized coefficient calculated through the following formula (5):

$$d = σ [ ( D - α ) / β ] \qquad ( 5 )$$

and wherein α is a mean value of the variable quantity (D), β is a standard deviation of the variable quantity (D), σ is a Sigmoid function.

In one or various embodiments, the material distribution simulation comprises generating a material distribution simulant with the target material via a finite element simulator (11), generating a rigid compress body, and exporting the material simulative parameter by simulating the rigid compress body compressing the material distribution simulant.

Preferably, the initial distribution simulant is generated by the finite element simulator (11) according to the optimal target parameter (P).

In preferred embodiments, please refer to FIG. 2B, the method further comprises a reduced model construction. In step S3, the reduced model construction comprises generating a Master curve via a Master curve generator (31) according to a compression data, wherein the compression data comprises Young's modulus, plateau stress and relative density; curve fitting a selected experimental structure to the Master curve via a material coefficient curve-fitting simulator (32) so as to obtain a material coefficient combination and exporting a material model coefficient combination; and assigning the material model coefficient combination to a single element via a target material generator (33) so as to obtain the target material, wherein the material model coefficient combination comprises a stiff material model coefficient combination, a soft material model coefficient combination or a combination thereof.

Preferably, the experimental structure comprises Gyroid, Primitive, F-RD, Fischer-Koch S, Diamond or I-WP, wherein the experimental structure is generated based on a metastructure model.

In one preferred embodiment, please refer to FIG. 2B, in step S3″, the compression data obtaining comprises generating the compression data via a compression experiment module (4), wherein a metastructure is generated by an experimental structure generator (41) according to a metastructure model, and the metastructure is compressed via a structure compressor (42) so as to obtain the compress data, wherein the metastructure is equal to or different from the experimental structure.

Preferably, the experimental structure comprises Gyroid, Primitive, F-RD, Fischer-Koch S, Diamond or I-WP, wherein the experimental structure is generated based on a metastructure model.

In particular, the metastructure model comprises a Triply Periodic Minimal Surface model (TPMS model).

Preferably, please refer to FIG. 2B, as illustrated in step S4, the method further comprises designing a bio-inspired simulated distribution simulant via a material design module (5) according to a second optimal target parameter customized by a user, wherein the second optimal target parameter is customized with the material simulative parameter comprising strain energy (SE), reaction force (RF), average mises stress (ST) or a combination of two or more thereof.

In order to specifically illustrate that the smart bio-inspired material design platform (100) and method thereof provided in the present invention can realize target-oriented material design with high efficiency and excellent precision, the technical efficacy of the platform (100) is further verified below through practical examples.

### Example 1: Shoe Midsole Design

The shoe midsole design requires to take at least three structures of different targets. Shown in FIG. 9A to 9C are 3 material distribution simulants and corresponsive simulative strain distribution diagrams. The material distribution simulants were customized through the smart bio-inspired material design platform (100) according to 3 different sets of optimal target parameters.

In the first place, as shown in FIG. 9A, the material distribution simulant 1 was concerned with only a single target function, and the material distribution simulant 1 aimed to design a shoe midsole for the arch, and the design space was 8×8. The target structure was anticipated to present high support capacity. Therefore, the optimal target parameter was customized to maximize the reaction force (RF). Through reinforcement learning computation, stiff materials concentrated at the compression center so as to enhance the support capacity.

In the second place, as shown in FIG. 9B, the material distribution simulant 2 had the same design space as the material distribution simulant 1, but the material distribution simulant 2 regarded a multi-target design and aimed to design a midsole for the heel. The target structure was anticipated to absorb more strain energy. The excessive energy produced by heel bottom transmitting to the leg of the user could be diminished so that discomfort after long-time walking can be prevented. Moreover, excessive rebound force upon the structure due to over-concentrated stress could also be avoided so that the heel rebound force would not be transmitted to joints such as the knee. Therefore, the optimal target parameter was customized to maximize the strain energy (SE), and to minimize reaction force (RF) and average mises stress (ST) at the central part of the shoe midsole.

In the third place, as shown in FIG. 9C, the material distribution simulant 3 had a same design of 12×12, and was also designed to meet multiple targets. The material distribution simulant 3 aimed to design a shoe midsole for foot toe, and was anticipated to have a softer target structure. Therefore, stress concentration should be reduced and more strain energy-absorptive so as to protect foot toe which is structurally weaker. Thus, the optimal target parameter was customized to maximize strain energy (SE) and minimize the average mises stress (ST) at the central part of the shoe midsole.

Listed in TABLE 1 were the customized optimal target parameters of the aforementioned 3 different material distribution simulants.

TABLE 1

| | simulant 1 | simulant 2 | simulant 3 |
|---|---|---|---|
| Strain energy (SE) | — | Max | Max |
| Average mises stress (ST) | — | Min | Min |
| Reaction force (RF) | Max | Min | — |

Listed in TABLE 2 were the simulated material parameters of the aforementioned 3 different material distribution simulants which were simulated by the platform (100).

TABLE 2

| | simulant 1 | simulant 2 | simulant 3 |
|---|---|---|---|
| Strain energy (SE) | — | 1.01 | 3.83 |
| Average mises stress (ST) | — | 0.16 | 0.73 |
| Reaction force (RF) | 30.29 | 6.91 | — |

After the optimal material distribution of the 3 different material distribution simulants were confirmed, 3D printing models for each simulant were established with an experiment structure generator (41) program. These 3 designs were then manufactured by 3D printing and designated as test body 1 (simulant 1), test body 2 (simulant 2), and test body 3 (simulant 3), respectively. To validate whether the aforementioned designs satisfy the optimal target parameter in entity testing, in this example, a TM-142 testing machine was used for falling weight impact absorption test. The testing parameters included peak deceleration, rebound value, maximum dent depth, original thickness and thickness after impact.

As shown in TABLE 3, the primary reference was the peak deceleration (g) and the rebound value. The smaller the g value and the rebound value, the more energy the designed bio-inspired material structure can absorb, and the larger the g value, the more energy is rebounded. Validation was carried out on each test body manufactured above, and the g value of test body 1 was 8 and the rebound value was 2%, the g value of test body 2 was 7 and the rebound value was 2%, and the g value of test body 3 was 9 and the rebound value was 4%. The values of the three energy absorption performances were lower than those obtained in the conventional impact absorption test. For example, the g value in the conventional impact test is usually between 9 and 15. This indicates that the bio-inspired material structure designed through the platform (100) could achieve the purpose of absorbing more energy no matter under the setting of a single target parameter or multiple target parameters, and satisfied the optimal target parameter customization.

TABLE 3

| | test body 1 | test body 2 | test body 3 |
|---|---|---|---|
| Peak deceleration (g) | 80 m/s² (8) | 70 m/s² (7) | 90 m/s² (9) |
| Rebound value | 2% | 2% | 4% |
| Maximum dent depth | 9.5 mm | 4.5 mm | 5.0 mm |
| Original thickness | 22.0 mm | 22.7 mm | 22.6 mm |
| Thickness after impact | 13.9 mm | 7.0 mm | 10.2 mm |

In addition, the test body 1 aiming for a single target design concentrated the stiff materials at the center of the shoe midsole and was expected to have higher support capacity. As shown in TABLE 3, the thickness after impact of the test body 1 was 13.9 mm which was larger than both of the test body 2 and the test body 3 (7.0 mm and 10.2 mm respectively). Apparently, the test body 1 demonstrated better support capacity than both of the test body 2 and the test body 3, so the thickness after impact of the test body 1 was higher than the other two. On the other hand, the test body 1 had high support capacity, but during the impact test the test body 1 produced the largest maximum dent depth (9.5 mm) than the other two (4.5 mm and 5.0 mm, respectively). The test body 1 generated larger strain energy than the other two test bodies under stress during the weight impact, which met the target setting to neglect strain energy (SE) when customizing the parameters. While parameter setting of the test body 2 and the test body 3 included strain energy (SE) at parameter customization, less strain energy was generated during the impact test, which also met the anticipated target setting.

The smart bio-inspired material design platform provided in the present invention, combined with a deep learning framework and a set of standard design processes, can simulate the corresponding materials quite efficiently as long as the reward function model in the reinforcement learning model is clearly defined. The material distribution design can meet the material design requirements in various situations, and is not limited by the structure of the object corresponding to the simulation. It can design the arrangement and distribution of materials for object structures such as plane, three-dimensional, symmetrical or asymmetrical, and has a wide range of potential applications.

The reduced model construction is used in the present invention. Assigning the microstructure coefficient to the target material endows the target material with specific mechanical properties. In the material design involving complex structures, the reduced model can be used with subsequent symmetric or asymmetric simulation methods. With a fixed target material quantity, the simulation calculation time is greatly reduced so as to decrease the time cost of material design.

The smart bio-inspired material platform provided in the present invention specifically combines the bio-inspired material structure, finite element simulation and deep learning framework to carry out the simulation design of bio-inspired materials. As shown in FIG. 10, the potential application level of the present invention is quite extensive. As long as the material parameters of a bio-inspired material structure can be obtained, such as compression, tension, extension, buckling or other parameters, the user can customize the optimal target parameters according to the needs of the back-end products, and further uses the platform to simulate a bio-inspired material structure distribution meeting the optimal the target parameters, and then the products are manufactured according to the simulation results.

The smart bio-inspired material design platform provided in the present invention, through parameter fitting and model construction, successfully reduces the phenomenon of reaction force and stress concentration when the product is impacted, and shows that it is beneficial to the product material design of complex microstructure. As shown in FIG. 10, the platform can be anticipated to be widely used in various industrial fields, such as shoe sole design, integrated sandals, badminton racket handle design, helmet design, golf structure design, military defense, aerospace, automotive industry or other industrial fields.

What is claimed is:

1. A smart bio-inspired material design platform comprising:
a material distribution simulating module configured to execute a target material distribution simulation so as to obtain a material simulative parameter; and
a reinforcement learning module, communicating to the material distribution simulating module, configured with a deep learning framework for executing a reward function model to compute a reward value of an iterative distribution simulant of another target material according to an optimal target parameter (P), and evaluating whether the iterative distribution simulant meets the optimal target parameter (P), wherein:
the target material comprises a stiff material and a soft material, and each material is set with different material model coefficient combinations;
the optimal target parameter is set according to the material simulative parameter comprising strain energy (SE), reaction force (RF), average mises stress (ST) or a combination of two or more thereof.

2. The smart bio-inspired material design platform as claimed in claim 1, wherein the reward function model comprises:
changing an initial distribution simulant into the iterative distribution simulant so as to obtain a variable quantity (D) based on the deep learning framework;
assigning the initial distribution simulant an initial reward value (q1) and assigning the iterative distribution simulant an end reward value (q2) so as to compute a standardized reward value (Q); and
the deep learning framework evaluates whether the iterative distribution simulant meets the optimal target parameter (P) according to the standardized reward value (Q).

3. The smart bio-inspired material design platform as claimed in claim 2, wherein the standardized reward value (Q) is computed according to the following formula:
$$Q = [ q 1 q 2 ] * d * P ,$$
wherein d is a standardized coefficient calculated through the following formula:
$$d = σ [ ( D - α ) / β ] ,$$
and wherein α is a mean value of the variable quantity (D), and β is an standard deviation of the variable quantity (D), and σ is a Sigmoid function.

4. The smart bio-inspired material design platform as claimed in claim 1, wherein the material simulative parameter further comprises rigidity modulus, elasticity modulus, shear elasticity modulus, strain force, modulus of deformation, or modulus of strain variable.

5. The smart bio-inspired material design platform as claimed in claim 1, wherein the material distribution simulating module comprises:
a finite element simulator configured for generating a material distribution simulant with the target material, generating a rigid compress body, and exporting the material simulative parameter by simulating rigid compress body compressing the material distribution simulant.

6. The smart bio-inspired material design platform as claimed in claim 1, comprising a reduced model construction module communicating to the material distribution simulating module for constructing the target material, wherein the reduced model construction module comprises:
a Master curve generator for generating a Master curve according to a compression data, wherein the compression data comprises Young's modulus, plateau stress and relative density;
a material coefficient curve-fitting simulator for curve fitting a selected experimental structure to the Master curve so as to obtain a material coefficient combination and output a material model coefficient combination; and
a target material generator for assigning the material coefficient combination to a single element so as to obtain the target material, wherein the material model coefficient combination comprises a stiff material model coefficient combination, a soft material model coefficient combination or a combination thereof.

7. The smart bio-inspired material design platform as claimed in claim 6, wherein the experimental structure comprises Gyroid, Primitive, F-RD, Fischer-Koch S, Diamond or I-WP, or the experimental structure is generated based on a metastructure model, wherein the metastructure model comprise Triply Periodic Minimal Surface model (TPMS model).

8. The smart bio-inspired material design platform as claimed in claim 1, further comprising a compression experiment module connecting to the reduced model construction module, wherein:
the compression experiment module comprises an experimental structure generator for generating a metastructure based on a metastructure model, and an structure compressor for compressing the metastructure so as to obtain the compression data, and wherein the metastructure is equal to or different from the experimental structure.

9. The smart bio-inspired material design platform as claimed in claim 1, further comprising a material design module communicating to the reinforcement learning module for designing a bio-inspired simulated distribution simulant according to a second optimal target parameter customized by a user, wherein the second optimal target parameter is set by referring to the material simulative parameter comprising strain energy (SE), reaction force (RF), average mises stress (ST) or a combination of two or more thereof.

10. A method for designing smart bio-inspired material, comprising:
executing a target material distribution simulation with a material distribution simulating module so as to obtain a material simulative parameter, wherein the target material comprises a stiff material and a soft material, and each material is set with different material model coefficient combinations; and
executing a reward function model with a deep learning framework configured to a reinforcement learning module for computing a reward value, and evaluating whether an iterative distribution simulant meets the optimal target parameter (P), wherein the reward value is computed corresponding to an iterative distribution simulant of another target material according to an optimal target parameter (P), wherein the optimal target parameter is set by referring to the material simulative parameter comprising strain energy (SE), reaction force (RF), average mises stress (ST) or a combination of two or more thereof.

11. The method as claimed in claim 10, wherein the material simulative parameter further comprises rigidity modulus, elasticity modulus, shear elasticity modulus, strain force, modulus of deformation, or modulus of strain variable.

12. The method as claimed in claim 10, wherein the reward value computation comprises:
changing an initial distribution simulant into the iterative distribution simulant via the reward function model so as to obtain a variable quantity (D) based on the deep learning framework;
assigning the initial distribution simulant an initial reward value (q1) and assigning the iterative distribution simulant an end reward value (q2) so as to compute a standardized reward value (Q); and
evaluating whether the iterative distribution simulant meets the optimal target parameter (P) according to the standardized reward value (Q) via the deep learning framework.

13. The method as claimed in claim 12, wherein the standardized reward value (Q) is computed according to the following formula:
$$Q = [ q 1 q 2 ] * d * P ,$$
wherein d is a standardized coefficient calculated through the following formula:
$$d = σ [ ( D - α ) / β ] ,$$
and wherein α is a mean value of the variable quantity (D), and β is a standard deviation of the variable quantity (D), σ is a Sigmoid function.

14. The method as claimed in claim 10, wherein the material distribution simulating comprises:
generating a material distribution simulant with the target material via a finite element simulator, generating a rigid compress body, and
exporting the material simulative parameter by simulating the rigid compress body compressing the material distribution simulant.

15. The method as claimed in claim 10, wherein the material distribution simulating module comprises a finite element simulator configured for generating the initial distribution simulant by arranging the target material according to the optimal target parameter (P).

16. The method as claimed in claim 10, comprising a reduced model construction, wherein the reduced model construction comprises:
generating a Master curve via a Master curve generator according to a compression data, wherein the compression data comprises Young's modulus, plateau stress and relative density;
curve fitting a selected experimental structure to the Master curve via a material coefficient curve-fitting simulator so as to obtain a material coefficient combination and exporting a material model coefficient combination; and
assigning the material coefficient combination to a single element via a target material generator so as to obtain the target material, wherein the material model coefficient combination comprises a stiff material model coefficient combination, a soft material model coefficient combination or a combination thereof.

17. The method as claimed in claim 16, wherein the experimental structure comprises Gyroid, Primitive, F-RD, Fischer-Koch S, Diamond or I-WP, or the experimental structure is generated based on a metastructure model, wherein the metastructure model comprise Triply Periodic Minimal Surface model (TPMS model).

18. The method as claimed in claim 16, wherein the compression data obtaining comprises generating the compression data via a compression experiment module.

19. The method as claimed in claim 18, wherein the compression data generating comprises:
generating a metastructure according to a metastructure model via an experimental structure generator; and
compressing the metastructure via a structure compressor so as to obtain the compress data, wherein the metastructure is equal to or different from the experimental structure.

20. The method as claimed in claim 10, further comprising designing a bio-inspired simulated distribution simulant via a material design module according to a second optimal target parameter customized by a user, wherein the second optimal target parameter is set by referring to the material simulative parameter comprising strain energy (SE), reaction force (RF), average mises stress (ST) or a combination of two or more thereof.
